(19) United States
(12) Patent Application Publication
(10) Pub. No.: US 2006/0266357 A1
(43) Pub. Date: Nov. 30, 2006

(54) Oxygen concentrator with variable ambient pressure sensing control means

(75) Inventors: Norman R. McCombs, Tonawanda, NY (US); Robert Bosinski, West Seneca, NY (US); Michael R. Valvo, East Aurora, NY (US); Michael A. Chimiak, Williamsville, NY (US)

Correspondence Address: HISCOCK & BARCLAY, LLP, 2000 HSBC PLAZA, ROCHESTER, NY, 14604-2404, US

(73) Assignee: AirSep Corporation

(21) Appl. No.: 11/441,603

(22) Filed: May 25, 2006

Related U.S. Application Data
(63) Continuation-in-part of application No. 11/247,101, filed on Oct. 11, 2005.
(60) Provisional application No. 60/617,833, filed on Oct. 12, 2004.
(60) Provisional application No. 60/669,323, filed on Apr. 7, 2005.

(30) Foreign Application Priority Data
Oct. 11, 2005 (WO) PCT/US05/36273

(51) Int. Cl. A61M 16/00; A62B 7/04; A62B 7/00

(52) U.S. Cl. 128/204.26; 128/204.18; 128/204.21; 128/204.23

(57) ABSTRACT

An oxygen concentrator for delivering consistent doses of an oxygen concentrated gas to a user by adjusting the delivery time according to the ambient air pressure at the time of delivery.

### CROSS-REFERENCE TO RELATED APPLICATIONS

This application is a continuation-in-part of our co-pending U.S. patent application Ser. No. 11/247,101, McCombs et al, filed Oct. 11, 2005.

### FIELD OF THE INVENTION

This invention relates to the production of gases and the regulation of their flow and, more specifically, the production of oxygen enriched gases and their delivery in pulse doses.

### BACKGROUND OF THE INVENTION

Gas flow regulators are well known to be used in conjunction with gas supply sources such as high pressure oxygen tanks or other similar oxygen sources to supply oxygen enriched gases, for example, to persons requiring supplemental oxygen. Oxygen control devices have been developed that conserve such an oxygen supply by limiting its release only during useful times such as, for example, during the inhalation period of the person's breathing cycle. In such a device, drops in pressure are caused by inhalation which, in turn, activates the oxygen flow.

It also is known that the only air or oxygen usefully absorbed by the lungs is that oxygen inhaled at the initial or effective stage of inhalation or inspiration. The air or oxygen inhaled in the latter stage of inhalation is usually exhaled before it can be absorbed by the lungs. To take advantage of this phenomenon, a device may conserve oxygen supplies even more by actuating the flow of gas upon initial inhalation but also terminating the flow of oxygen after the effective stage. It is known, with such devices, to control the effective flow rate of the oxygen, according to the user's needs, by increasing or decreasing the activation time during each inhalation cycle.

One such combination pressure regulator and conservation device is disclosed in co-owned U.S. Pat. No. 6,427,690 to McCombs et al, issued Aug. 6, 2002, the entire disclosure of which is incorporated by reference herein. The device may conveniently be positioned directly on an oxygen tank (containing oxygen or an oxygen mixture in gas or liquid form), or connected to the wall outlet of a master oxygen system for connection directly to the tank or outlet. Contained within the device is an oxygen pressure regulator, a power supply or external power supply connection and a control circuit to control the effective dose of oxygen by control of the interval(s) and time(s) of the oxygen flow during every inhalation stage, during selectable, alternate inhalation cycles, or by a continuous supply of oxygen.

The conservation device may contain a first chamber to control the pressure of the supplied oxygen by a regulator spring and piston and may also contain a second or oxygen volume chamber in fluid connection with the first chamber. The second chamber is provided to maintain a predefined volume or “bolus” of oxygen at the pre-set pressure, and from which the oxygen is delivered through a tube to a user upon actuation of a valve operated by a control circuit. To actuate the valve in response to inhalation by the user, as disclosed for example in the foregoing patent, the control circuit includes a pressure sensing transducer that will sense a reduction in pressure caused by the inhalation and thus open the valve for a pre-programmed or otherwise suitable time.

In addition to the conservation device disclosed in U.S. Pat. No. 6,427,690, a portable oxygen concentrator has also been developed and which operates on pressure swing adsorption, or PSA, principles and includes an integral oxygen conservation device, as disclosed in co-owned U.S. Pat. No. 6,764,534, McCombs et al, issued Jul. 20, 2004, the entire disclosure of which is incorporated by reference. Furthermore, such an oxygen concentrator described in that patent is able to deliver, at the initial stage of inhalation, a product gas with a high oxygen concentration (e.g., up to about 95% oxygen) produced by the PSA components of the concentrator, equivalent therapeutically to continuous flow rates of at least up to 5 liters per minute (LPM).

The desired mode of operation is determined by positioning a mode control switch to the desired operating mode position. If the conservation device is a separate device, it is attached either to an oxygen tank or the outlet of a PSA apparatus, and the valve on the oxygen supply tank is then opened or the PSA apparatus turned on. In the normal intermittent operating mode, selector switches are used to select one of several operating settings to indicate the equivalent flow rate of the supplied oxygen, e.g., from 1-5 LPM. The oxygen delivery device, such a nose cannula, is then attached by its connecting tube to the outlet on the conservation device.

### SUMMARY OF THE INVENTION

The present invention provides an apparatus that is able to produce a product gas having a high concentration of a desired product gas or gases, such as oxygen, with the ability to control more accurately the amount of product gas supplied to a user as based on the ambient air pressure and preferably only on initiation of demand. This invention comprises a compressed product gas (e.g. oxygen) source or other such product gas producing means, such as a pressure swing adsorption (PSA) apparatus or vacuum pressure swing adsorption apparatus (VPSA), and a delivery control assembly to determine the length of time to supply the more accurate amount of product gas to the user by reference to the altitude or ambient air pressure at which the apparatus is in use.

As applied to an oxygen producing device, for example, the delivery control assembly serves two primary functions. First, since most oxygen normally inhaled is immediately exhaled and unused, the delivery control assembly provides a pulse dose of oxygen-rich gas only when it will be most efficiently utilized by the person inhaling it, thus minimizing unnecessary waste of the oxygen-rich product gas. This more efficient use of the oxygen supplied is very advantageous in minimizing the capacity requirements of the oxygen source, such as a compressed bottle or PSA apparatus. Reduced capacity requirements may translate to smaller, lighter, quieter and less expensive oxygen-rich gas production devices.

Second, the delivery control assembly, according to this invention, serves to ensure that its owner receives for any given flow setting a substantially constant quantity of oxygen during every inhalation. Because of the Ideal Gas Law, PV=nRT, it cannot be assumed that this amount will always be constant because the number of oxygen molecules in each dose will depend upon a number of factors, including, for example, the gas pressure of the ambient air drawn into the apparatus, and the pressure and temperature of the enriched gas within the apparatus at the time of inhalation.

The invention as disclosed in co-pending U.S. patent application Ser. No. 11/247,101 uses sensors that read, for example, real-time system operating pressures and/or temperatures, and converts the analog outputs of the sensors to digital signals to control the pulse dose through the use of a microprocessor in a micro-electronic control circuit. The present invention is a further improvement by which the control circuit also determines the proper pulse dose by taking into account either the actual altitude at which the apparatus is being used or in an atmosphere controlled environment such as the pressurized cabin of an aircraft. The ambient pressure may be either the actual pressure as determined by an altimeter device or the like, or by a selector switch having a number of predetermined settings approximating the altitude of selected geographic regions or elevations. The invention may be used in such apparatus whether or not the apparatus incorporates the inventions disclosed in U.S. patent application Ser. No. 11/247,101.

### BRIEF DESCRIPTION OF THE DRAWINGS

The above-mentioned and other features and advantages of this invention, and the manner of attaining them, will become apparent and be better understood by reference to the following description of several embodiments of the invention in conjunction with the accompanying drawings, wherein:

FIG. 1 is a schematic view of a Pressure Swing Adsorption (PSA) apparatus in which the invention may be incorporated;

FIG. 2 is a partial schematic view illustrating the control assembly for the first embodiment of the invention;

FIG. 3 is a block diagram of the control circuit for determining the length of the pulse dose based on the programmed ambient pressure;

FIG. 4 is a block diagram of the control circuit for a second embodiment of the invention, by which the pulse dose volume may be controlled for both ambient air pressure and for variations in temperature and/or pressure; and

FIG. 5 is a partial schematic view illustrating the control assembly for the second embodiment of the invention.

Corresponding reference characters indicate corresponding parts throughout the several views. The examples set out herein illustrate certain embodiments of the invention but should not be construed as limiting the scope of the invention in any manner.

### DETAILED DESCRIPTION

The invention described in this application may be used in either a PSA or VPSA apparatus, both of which are well known and described, for example, in U.S. Pat. Nos. 3,564,816; 3,636,679; 3,717,974; 4,802,899; 5,531,807; 5,755,856; 5,871,564; 6,524,370; and 6,764,534, among others. Both a PSA and a VPSA apparatus may include one or more adsorbers, each having a fixed sieve bed of adsorbent material to fractionate at least one constituent gas from a gaseous mixture by adsorption into the bed, when the gaseous mixture from a feed stream is sequentially directed through the adsorbers in a co-current direction. While one adsorber performs adsorption, another adsorber is purged of its adsorbed constituent gas. In a PSA apparatus, the purging is performed by part of the product gas being withdrawn from the first or producing adsorber and directed through the other adsorber in a counter-current direction. In a VPSA apparatus, the purging primarily is performed by a vacuum produced at the adsorber inlet to draw the purged gas from the adsorber. Once the other adsorber is purged, the feed stream at a preset time is then directed to the other adsorber in the co-current direction, so that the other adsorber performs adsorption. The first adsorber is then purged either simultaneously, or in another timed sequence if there are more than two adsorbers, all of which will be understood from a reading of the above described patents.

When, for example, such an apparatus is used to produce a high concentration of oxygen from ambient air for use in various applications, whether medical, industrial or commercial, air enters the apparatus typically containing about 78% nitrogen, 21% oxygen, 0.9% argon and a variable amount of water vapor. Principally, most of the nitrogen is removed by the apparatus to produce the product gas which, for medical purposes, for example, typically may contain at least about 80% and up to about 95% oxygen.

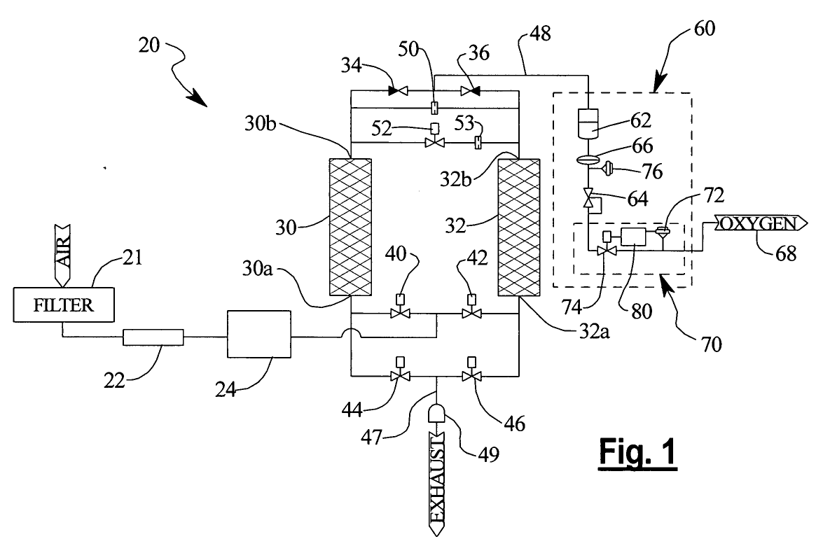

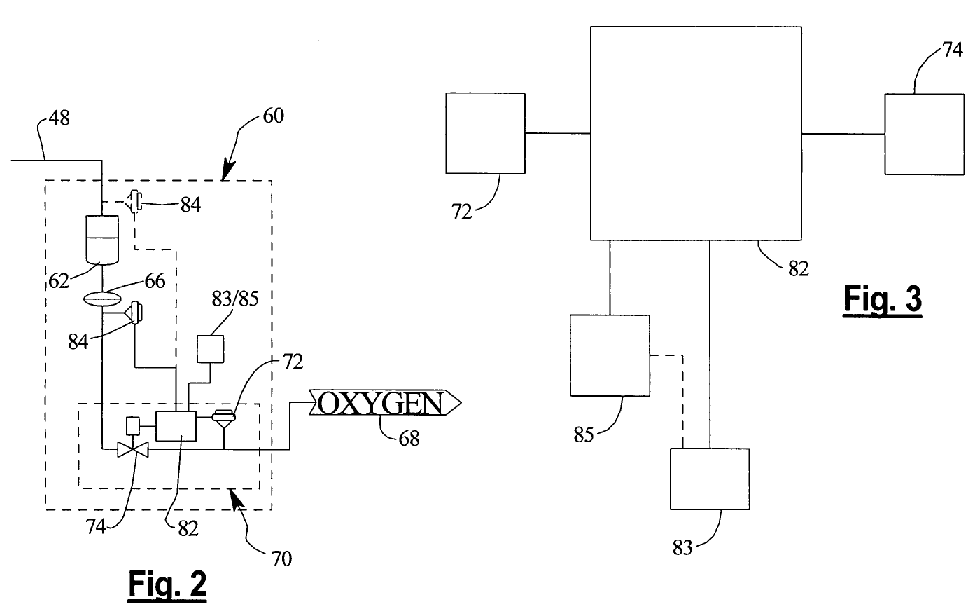

Referring to FIG. 1, ambient air is supplied to a PSA apparatus 20 through a filtered intake 21 and an intake resonator 22 to decrease the noise from the intake of the ambient air feed stream. The feed stream continues from the resonator 22 and is moved by a feed air compressor/heat exchanger assembly 24 alternatively to the first and second adsorbers 30, 32 through feed valves 40 and 42, respectively.

When the feed stream alternatively enters inlets 30a, 32a of adsorbers 30, 32 in a co-current direction, the respective adsorber fractionates the feed stream into the desired concentration of product gas. The adsorbent material used for the beds to separate nitrogen from the ambient air may be a synthetic zeolite or other known adsorber material having equivalent properties.

The substantial or usable portion of the oxygen enriched product gas generated from the ambient air flowing in the co-current direction sequentially in each one of the adsorbers 30, 32 is directed through the outlet 30b, 32b and check valve 34, 36 of the corresponding adsorber to a product manifold 48 and then to a delivery control assembly 60, as will be described. The balance of the product gas generated by each adsorber is timed to be diverted through a purge orifice 50, a properly timed equalization valve 52 and an optional flow restrictor 53 to flow through the other adsorber 30 or 32 in the counter-current direction from the respective outlet 30b, 32b and to the respective inlet 30a, 32a of the other adsorber to purge the adsorbed, primarily nitrogen, gases. The counter-current product gas and purged gases then are discharged to the atmosphere from the adsorbers through properly timed waste valves 44, 46, common waste line 47 and a sound absorbing muffler 49.

The control assembly 60, to which the usable portion of the produced gas is directed, typically includes a mixing tank 62 which also may be filled with synthetic zeolite and serves as a reservoir to store product oxygen before delivery to the user through an apparatus outlet 68 in the pulse dose mode, a pressure sensor 76 to monitor the pressure of the product gas at the mixing tank 62 (normally, for example, to monitor for extreme pressure levels and activate a warning signal), a piston-type pressure control regulator 64 to regulate the product gas pressure to be delivered to the user, an optional bacteria filter 66, and an oxygen delivery system 70 including a pulse dose transducer 72, the conservation unit 80 to be described, and a flow control valve 74. Delivery of the PSA generated oxygen concentrated gas from the mixing tank 62 to the user is controlled by the delivery system 70 as will be described.

A VPSA apparatus operates in similar fashion as the PSA apparatus of FIG. 1, except that the purge orifice 50 may be eliminated. In its stead, a vacuum pump is provided in the common waste line 47 to draw the waste nitrogen alternately from each of adsorber beds 30, 32 upon the timed opening of the respective waste valve 44, 46. The cycling of ambient air and operation of the feed and waste valves to produce the oxygen enriched product gas, as well as of supply of product gas to mixing tank 62 and the delivery of the product gas by conservation unit 80, otherwise are as described with respect to FIG. 1.

As described earlier, a conservation device delivers, when the patient inhales, a consistent and specific pulse dose of oxygen to the patient at preset times depending on the selected flow setting of the device and equivalent to a continuous flow rate. The product gas delivery pressure, as set by a pressure regulator, e.g., 64, together with the preset open time for an oxygen delivery demand valve, which may be a solenoid actuated flow control valve 74 as earlier described, generally determines the volume of the product gas delivered to the user. This technique, to open upon inhalation the demand valve for a certain amount of time to deliver the desired dose, may be used with cylinders of oxygen and in PSA or VPSA oxygen concentrators.

A pressure regulator is known in the prior art to be necessary when a conservation device is used with oxygen cylinders and with oxygen concentrators. Whatever the pressure in an oxygen tank, the regulator regulates the pressure down to approximately 20 psig to obtain a consistent pulse dose as the cylinder depressurizes over time. In a PSA apparatus, the cycle pressure can vary, e.g., from about 15 to about 26 psig, and the regulator regulates the pressure at the demand valve, e.g., to approximately 10 psig. Similarly, the cycle pressure for a VPSA may vary e.g., from about −25 to about 10 psig and regulated at the demand valve to about 3 psig.

Additionally, the actual amount of oxygen to be delivered to a user of the apparatus will be a function of other factors, including the length of time that a valve is open, the operational temperature of the gas at the time it is being supplied, the altitude at which the apparatus is being used, and the breathing rate of the user. For example, at either or both a higher temperature or altitude, less oxygen will be delivered to a user for any given period of time. Similarly, less oxygen will be delivered to the user at lower pressures caused by, among other things, more rapid breathing rates that will affect the product gas pressure. Unlike the known prior art, the invention described here comprises an oxygen concentrator 20 that is able to control the pulse dose time in order to deliver a substantially consistent and predetermined quantity of oxygen based the pressure of the ambient air drawn into the apparatus, as opposed to fixed, predetermined delivery times in which the actual quantity of oxygen will vary based on the Ideal Gas Law. In addition to basing the time on ambient air pressure, the apparatus may also incorporate the inventions disclosed in co-pending U.S. patent application Ser. No. 11/247,101.

According to the invention, the pulse dose may be controlled by a system incorporating an altimeter or similar device that includes a pressure transducer to read the pressure of the ambient air and to feed that reading as a signal, converted to a digital signal if analog, into a microprocessor to adjust the pulse dose time according to the ambient air pressure. Preferably, the signal causes the microprocessor to access a preprogrammed data table of pressure/dose time settings to account for ambient air pressure adjustments caused by the specific compressor used in the apparatus. In one embodiment of this invention, the length of the pulse dose to deliver the desired quantity of oxygen is dependent on the ambient air pressure. In a second embodiment, the length of the pulse dose is determined at inhalation both by the ambient air pressure and by the actual temperature and/or actual system pressure of the product gas preferably but not necessarily at or near the mixing tank 62.

The first embodiment of this invention takes advantage of that fact that the amount of oxygen that is delivered by the invention is a function of ambient air pressure. As can be seen in TABLE 1, which illustrates a concentrator having three flow selector settings, when the ambient air pressure sensing circuit reads a pressure of between 10.5 psia and 15 psia, the demand valve 74 remains open for a time period as defined for that particular pressure. This time period is received by the microprocessor from the appropriate data table. For example, if the ambient air pressure is about 14.7 psia, the demand valve 74 parameters in the look-up table for that particular pressure for three flow settings are accessed. Generally, a decrease in ambient air pressure results in an increase in the time that the demand valve 74 remains open, or the Pulse Dose Time. When the transducer 72 first senses inhalation, the microprocessor will obtain a reading of the atmospheric pressure from the ambient air pressure sensor. It will then use a lookup data table in the microprocessor's memory to determine the correct amount of time to open the demand valve. The Table 1 lists example valve open times for various atmospheric pressures for a portable oxygen concentrator.

TABLE 1

| | Pulse Dose Time (ms) | | | | | | | | | | |
|---|---|---|---|---|---|---|---|---|---|---|---|
| | Pressure (psia) | | | | | | | | | | |
| Flow Setting | 15.0 | 14.7 | 14.5 | 14.0 | 13.5 | 13.0 | 12.5 | 12.0 | 11.5 | 11.0 | 10.5 |
| 1 | 59 | 60 | 61 | 63 | 65 | 68 | 71 | 74 | 77 | 80 | 84 |
| 2 | 118 | 120 | 122 | 126 | 131 | 136 | 141 | 147 | 153 | 160 | 168 |
| 3 | 176 | 180 | 182 | 189 | 196 | 204 | 212 | 221 | 230 | 241 | 252 |

Two possible ambient air pressure sensors are a Honeywell ASDX015A24R or a Bosch SMD085. Both of these are amplified and temperature compensated absolute pressure transducers with a 0-5 VDC output.

Alternatively, a selector switch can be used in place of an atmospheric pressure transducer. The user will adjust the switch to a preprogrammed elevation or location, as for example by use of the following, albeit abbreviated, data Table 2.

TABLE 2

| | Valve Open Times (ms) | | |
|---|---|---|---|
| | | | Pressurized |
| Flow | Sea Level | Denver, CO | Aircraft |
| Setting | (14.7 psia) | (12.2 psia) | (10.9 psia) |
| 1 | 60 | 73 | 81 |
| 2 | 120 | 145 | 162 |
| 3 | 180 | 217 | 243 |

Both an ambient air pressure sensor and a selector switch may be used, in which case the selector switch may have an additional setting to engage the ambient air pressure sensor as shown in by dotted lines in FIG. 3. According to this alternative, the microprocessor may activate a warning signal if the atmospheric pressure transducer is not functioning normally, in which case the user can immediately move the selector switch to one of the preprogrammed settings shown in Table 2.

In a second embodiment of the invention, the apparatus may also adjust the pulse time according to the system operating pressure and/or the temperature of the enriched oxygen gas at the time of delivery. For that purpose, the apparatus will include the inventions described in co-pending U.S. patent application Ser. No. 11/247,101, the entire disclosure of which is incorporated by reference. In that case, each of the ambient air pressure, system operating pressure and delivery gas temperature all are inputs to the microprocessor. The data tables to be accessed are then expanded to account for all three inputs from the three environmental ranges and for all of the flow settings for the apparatus.

As disclosed in co-pending U.S. patent application Ser. No. 11/247,101, the power efficiency of the apparatus can be improved if the compressor/heat exchanger assembly 24 is programmed to operate at a different speed for each flow setting, at speeds of about 1750 rpm for the equivalent continuous flow rate of 1 LPM, about 2500 rpm for the equivalent continuous flow rate of 2 LPM, and about 3200 rpm for the equivalent continuous flow rate of 3 LPM.

FIG. 3 is a block diagram representing the control circuit according to the first embodiment. For illustrative purposes, the figure includes only the pressure transducer 72 to sense inhalation, the ambient air pressure sensor 83, a selector switch 85, the microprocessor 82 containing the look-up table for ambient air pressure, and the demand valve 74. Generally, the inhalation pressure transducer 72 serves to detect a change in pressure which would indicate the start of the inhalation cycle. Upon sensing inhalation, the inhalation pressure transducer 72 transmits a signal suitable for processing by the microprocessor 82, which in turn accesses its look-up table for atmospheric pressure, and signals the demand valve 74 to be actuated for the appropriate length of time. While FIG. 3 provides a block diagram representative of the control circuit according to the present invention, details of the specific circuit elements and microprocessor logic can be determined by those skilled in the art and by reference, for example, to the circuit described in U.S. Pat. No. 6,764,534.

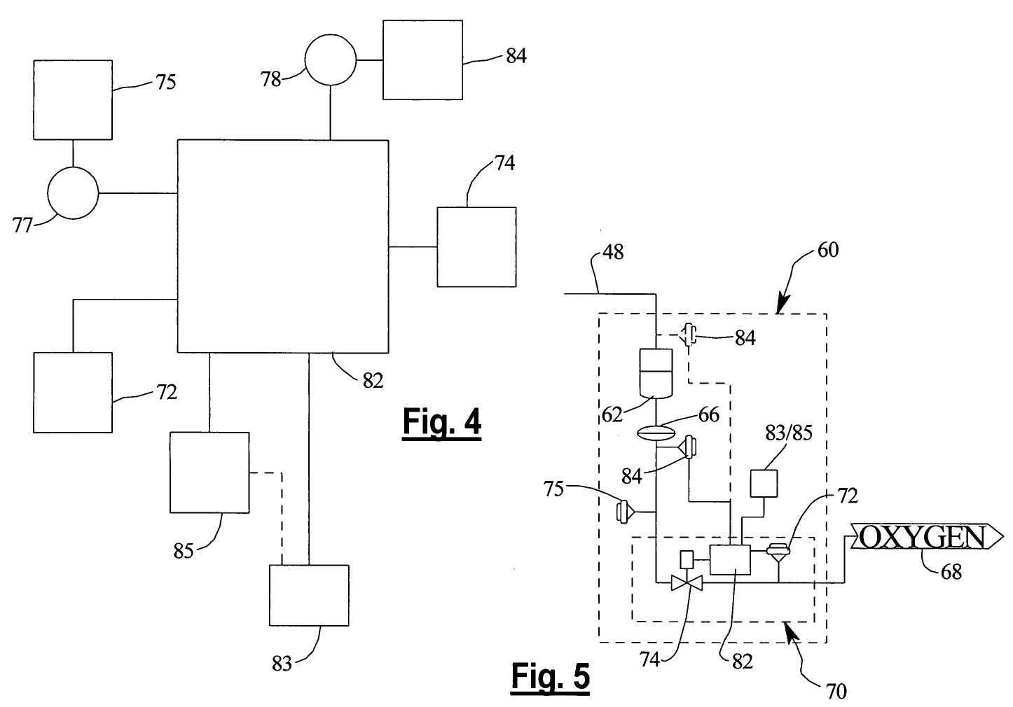

FIG. 4 and FIG. 5 are representative of the control circuit 60 according to the second embodiment of the present invention, by which the actual operating pressures and/or actual operating temperatures also are used to determine the dose of oxygen enriched product gas to be delivered to the user. For illustrative purposes, FIG. 4 is a block diagram that includes the pressure transducer 72 to sense inhalation, a temperature sensing circuit 77 for reading an analog signal of the temperature sensed by a temperature sensor or thermistor 75 at the point of inhalation and converting it to a digital signal, a pressure sensor 84 at the mixing tank the output of pressure sensor 84 if not a digital signal is converted to a digital signal by a pressure sensing circuit 78, ambient air pressure sensor 83, microprocessor 82 to read the three signals, and control demand valve 74 actuated by the microprocessor 82 in response to pressure transducer 72. Upon detection of inhalation by the pressure transducer 72, the microprocessor 82 reads the digital signals derived from the temperature sensor 75, pressure sensor 84 and ambient air pressure sensor 83 respectively. It is also possible that the three sensors, although depicted as separate instruments, may be in a single monitoring device capable of reading all three parameters.

The microprocessor 82 is pre-programmed to contain all of the data tables which define the length of time that the demand valve 74 is to remain open, as described above, for each of the temperature ranges and given system pressure range for each altitude setting, or in any other permutation of those parameters. Based on the flow selector setting and on receipt of the digital signals derived from the three sensors, the microprocessor 82 refers to the appropriate data table(s) which then actuates the demand valve 74 according to the time value listed in the data table. While FIG. 4 provides a block diagram representative of the control circuit according to the second embodiment of the present invention, details of the specific circuit elements and microprocessor logic can be determined by those skilled in the art and by reference, for example, to the circuit described in U.S. Pat. No. 6,764,534.

As it has been described that the amount of oxygen administered is a function of pulse dose time as related to the pressure of the oxygen in the system, it should be reasonably clear that oxygen pressure, which would thereby determine pulse dose time, is dependent upon all three ambient conditions, such as atmospheric pressure and enriched gas temperature as well as the changes in pressure inherent to a PSA or VPSA apparatus.

Because the flow selector settings (in LPM) in principle are common in the previous embodiments, there still remains a human element in deciding the specified pulse dose. In the first embodiment, the data essentially used to calculate pulse dose values are in terms of a correction factor to a nominal pulse dose of 200 ms. In the second embodiment, however, a nominal pulse dose is no longer used as a base point, but instead the dose is determined by the microprocessor calculating the actual pulse dose times based on actual ambient and system pressures and temperature. Thus, in the second embodiment, the microprocessor 82, continuously receives baseline temperature and pressure information derived from the temperature sensor 75 and pressure sensors 83, 84. It is preferable that these values be constantly be measured as the microprocessor 82 may need to average a relatively short time history of those values to adjust baseline pressures and temperatures over time during use of the apparatus. From this baseline set of values, the microprocessor may know the proper baseline pulse dose from a designated table stored in the microprocessor memory. When the inhalation pressure transducer 72 senses a pressure drop due to inhalation, the microprocessor 82 senses this and reads the volume pressure at that moment in time via the pulse dose transducer 84. This value will allow the microprocessor locate a correction factor from an independent set of tables which are based on the continuously changing volume pressure in a PSA or VPSA cycle, and apply that correction to produce the final required pulse dose.

While the invention has been described with reference to particular embodiments, it will be understood by those skilled in the art that various changes may be made and equivalents may be substituted for elements thereof without departing from the scope of the invention. Many modifications may be made to adapt a particular situation or material to the teachings of the invention without departing from the scope of the invention. For example, with a microprocessor having sufficient memory, it is possible to determine the length of the pulse dose by integrating the actual temperatures and pressures. In addition, the invention may incorporate the many of the useful features of the concentrator as disclosed in U.S. Pat. No. 6,764,534.

Therefore, it is intended that the invention not be limited to the particular embodiments disclosed as the best mode contemplated for carrying out this invention, but that the invention will include all embodiments falling within the scope and spirit of the appended claims.

What is claimed is:

1. Apparatus comprising means for producing a product gas having a high concentration of oxygen, means for controlling a desired amount of the product gas delivered through an outlet to a user only on initiation of demand, means for delivering the product gas to the outlet, and means for determining the ambient air pressure at which the concentrator is being used, and the control means comprising means utilizing the determined ambient air pressure for setting the length of time to supply substantially that amount of product gas to the user.

2. Apparatus according to claim 1 in which the producing means generates the product gas from ambient air at predictable variations in ambient air pressure at specified geographic altitudes and/or controlled atmospheres, and the control means comprises a manually adjustable selector switch from which to select from a number of predetermined altitudes and/or controlled atmospheres.

3. Apparatus according to claim 2, in which the control means comprises a set of look-up tables containing the lengths of time specific to the selected altitudes.

4. Apparatus according to claim 3, in which the look-up tables are resident in a microprocessor.

5. Apparatus according to claim 2 in which the control means further comprises an ambient air pressure sensor to measure the actual ambient air pressure, and in which the selector switch has a further setting for enabling the length of time to be determined by the sensor measurement.

6. Apparatus according to claim 2, in which the control means further comprises means for determining the lengths of time to supply the product gas by reference to operating pressures and/or temperature of the product gas at the time of inhalation.

7. Apparatus according to claim 1 in which the control means comprises an ambient air pressure sensor to measure the actual ambient air pressure.

8. Apparatus according to claim 7, in which the control means comprises a set of look-up tables containing the lengths of time specific to the ambient air pressure measurements .

9. Apparatus according to claim 8, in which the look-up tables are resident in a microprocessor.

10. Apparatus according to claim 7, in which the control means further comprises means for determining the lengths of time to supply the product gas by reference to operating pressures and/or temperature of the apparatus at the time of inhalation.

11. Apparatus according to claim 1, in which the control means further comprises means for determining the lengths of time to supply the product gas by reference to operating pressures and/or temperature of the apparatus at the time of inhalation.

12. A method for determining a desired dose of a product gas generated by an apparatus having means for producing a high concentration of oxygen and delivering the gas through an outlet to a user, comprising the steps of sensing inhalation by the user, determining the altitude of the apparatus at the time inhalation is sensed, and delivering the desired dose of product gas to the user in a length of time based on the determined altitude.

13. The method of claim 12 in which the step of delivering the desired dose of product gas is preceded by the steps of calculating the times required to deliver the desired dose of product gas for specific altitudes, and producing a reference table of the calculated times to be accessed for the delivery step.

14. In an apparatus having means for producing a desired dose of product gas having a high concentration of oxygen and delivering the gas through an outlet to a user, a method comprising the steps of presetting a number of estimated ambient air pressures based on selected altitudes and/or controlled atmospheres at which the apparatus may be used, selecting the one of the preset ambient air pressures at which the apparatus will be used, sensing inhalation by the user, determining the operating pressure and/or temperature of the product gas at the time at which the inhalation is sensed, and delivering the gas to the user for a length of time based on the preset ambient air pressure and on the operating pressure and/or temperature.
